(19) United States
(12) Patent Application Publication
(10) Pub. No.: US 2026/0220775 A1
(43) Pub. Date: Jul. 30, 2026

(54) APPARATUS, METHOD, AND NON-TRANSITORY COMPUTER READABLE MEDIUM FOR PERFORMING TRAINING PROCESSING OF ESTIMATION MODEL

(71) Applicant: Yokogawa Electric Corporation, Tokyo (JP)

(72) Inventor: Tomoya ITO, Tokyo (JP)

(21) Appl. No.: 19/457,148

(22) Filed: Jan. 22, 2026

(30) Foreign Application Priority Data
Jan. 30, 2025 (JP) 2025-014291

(51) Int. Cl. G06T 7/00; G06V 20/69; G16H 30/40

(52) U.S. Cl. CPC G06T 7/0012; G06V 20/695; G16H 30/40; G06T 2207/10056; G06T 2207/20036; G06T 2207/20081; G06T 2207/30024

(57) ABSTRACT

Provided is an apparatus including a processor, in which the processor calculates an individual feature value of each of a plurality of objects by using an image of each of the plurality of objects and a group label assigned to a group including the plurality of objects, and performs training processing of an estimation model which outputs, when an image of an object is input, an estimation value of the individual feature value of the object by using the image and the individual feature value of each of the plurality of objects.

### BACKGROUND

### 1. TECHNICAL FIELD

The present invention relates to an apparatus, a method, and a non-transitory computer readable medium for performing training processing of an estimation model.

### 2. RELATED ART

Patent Document 1 describes that "The present invention relates to a method for supervised classification for classifying cells contained in images ... in images obtained with microscopes" (paragraph 0001 of Patent Document 1) and "With reference to FIG. 2, this learning step allows the accuracy of the classification to be improved by computing a prototype for a supervised classifier resulting from cells labeled by an expert, and such a prototype is calculated by minimizing a classification error function representing unfavorable classification results" (paragraph 0080 of Patent Document 1).

Patent Document 2 describes that "In a first step 102, an image analysis system 200 ... receives a plurality of digital tissue images 212. For example, each tissue image can depict a whole-slide tissue sample taken from a patient, for example, a cancer patient” (paragraph 0189 of Patent Document 2), "Next, in step 104, the image analysis system splits each received image into a set of overlapping or non-overlapping image tiles 216" (paragraph 0190 of Patent Document 2), "Next, in step 106, the image analysis system computes, for each of the tiles, a feature vector 220" (paragraph 0192 of Patent Document 2), and "The trained and instantiated MIL-program then processes the tiles of the received digital tissue images at test time for classifying the received tissue images" (paragraph 0193 of Patent Document 2).

Patent Document 3 describes that “The disclosure presented herein provides a method for quantifying viable cells and particulate cell impurities in a cell-based product sample. The method is implemented on a convolutional neural network (CNN) that learns to classify flow-imaging microscopy (FIM) images. The CNN learning is accomplished by using a training set of classified images of viable cells and different types of impurities” (paragraph 0001 of Patent Document 3).

Patent Document 4 describes that "The invention of the present disclosure extends and modifies state-of-the-art technology in experimental high-throughput flow imaging microscopy, flow cytometry, machine learning, and computational statistics. The present invention enables the ability to classify experimental images into predetermined classes and/or label the observation results as an a priori known or a priori unknown “fault”. The “fault” means that the observation results are statistically unlikely to have come from a measured reference population of responses. As generally illustrated in FIG. 1, the present invention may include a multi-component system to capture high-throughput flow imaging microscope and apply machine learning applications to such images and thereby achieve a classification of subject particles, cell, biomolecule, or another target” (paragraph 0038 of Patent Document 4).

### RELATED ART DOCUMENTS

### Patent Documents

Patent Document 1: Japanese Translation Publication of a PCT Route Patent Application No. 2015-508501

Patent Document 2: Japanese Translation Publication of a PCT Route Patent Application No. 2023-501126

Patent Document 3: Japanese Translation Publication of a PCT Route Patent Application No. 2024-517592

Patent Document 4: Japanese Translation Publication of a PCT Route Patent Application No. 2021-532350

### BRIEF DESCRIPTION OF THE DRAWINGS

FIG. 1 illustrates a configuration of a system 10 according to the present embodiment.

FIG. 2 illustrates an evaluation processing flow of the system 10 according to the present embodiment.

FIG. 3 illustrates an overall flow of training processing of the system 10 according to the present embodiment.

FIG. 4 is a detailed flow of the training processing of the system 10 according to the present embodiment.

FIG. 5 illustrates an example of a computer 2200 in which a plurality of aspects of the present invention may be embodied in whole or in part.

### DESCRIPTION OF EXEMPLARY EMBODIMENTS

The present invention will be described below through embodiments of the invention, but the following embodiments do not limit the invention according to the claims. In addition, not all combinations of features described in the embodiments are essential to a solution of the invention.

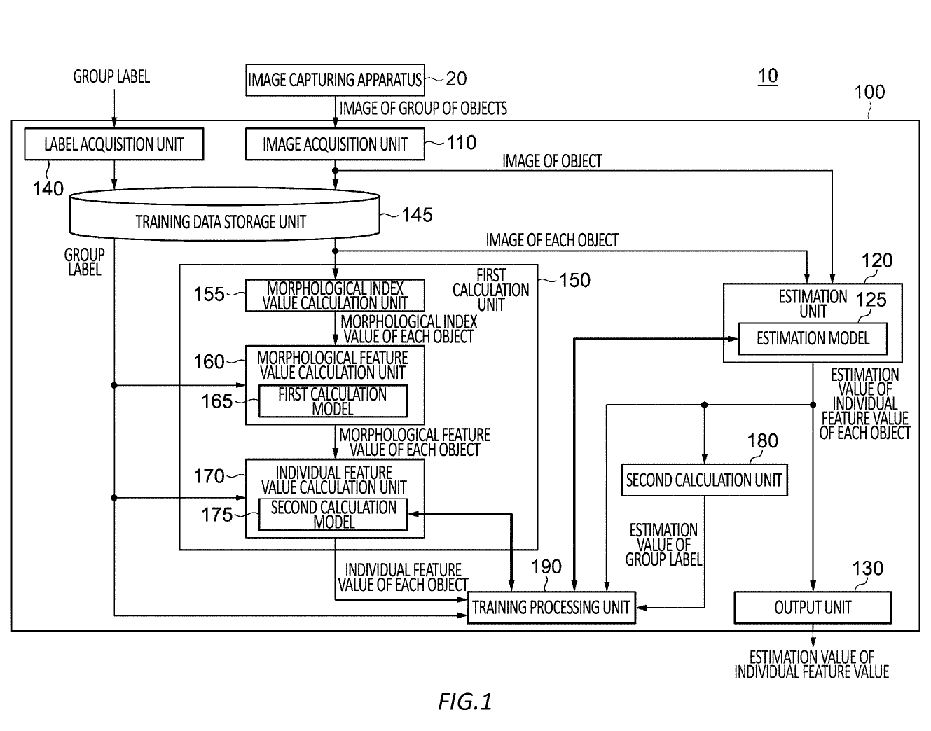

FIG. 1 illustrates a configuration of a system 10 according to the present embodiment. The system 10 includes an image capturing apparatus 20 and an apparatus 100. The image capturing apparatus 20 captures images of a group including a plurality of objects and supplies the images of the plurality of objects to the apparatus 100. The image capturing apparatus 20 may capture, as one image, an entire group including the plurality of objects, or may separately capture respective images of the plurality of objects in the group.

The plurality of objects may be of different types, and at least two or more of the objects may be of the same type. The object may be, for example, a cell, a microorganism, an animal, a plant, or another living body, a part of a living body, a virus, or the like. Alternatively, the object may be any object that individually has a shape and a property or feature, and that also has a property or feature as a collection of a plurality of objects.

In the present embodiment, a case where a group including a plurality of objects is a cell group, that is, for example, a sample including a plurality of cells or the like, and each object is a cell will be described as an example. When the object is a cell or a microorganism, the image capturing apparatus 20 may be a flow imaging apparatus. The flow imaging apparatus causes a liquid sample to flow through a thin flow channel such that a plurality of objects (cells, microorganisms) included in the sample pass through the flow channel one by one. Then, the flow imaging apparatus captures each individual of the objects flowing through the flow channel one by one, thereby capturing an image of each individual (such as an image of each cell) of the objects included in the sample. Accordingly, the image capturing apparatus 20 can capture an image of each of thousands to ten thousands of individuals, for example.

Alternatively, the image capturing apparatus 20 may be a microscope apparatus which captures an enlarged field of view of a sample. In this case, the image capturing apparatus 20 may output a captured image including an image of each individual of cells or microorganisms from an image of an entire field of view.

The apparatus 100 is connected to the image capturing apparatus 20. The apparatus 100 may have at least one of a function of training an estimation model which estimates a feature value of an object or a function of estimating the feature value of the object by using the estimation model. The apparatus 100 may be a computer such as a personal computer (PC), a tablet computer, a smartphone, a workstation, a server computer, or a general-purpose computer, or may be a computer system in which a plurality of computers is connected. Such a computer system is also a computer in a broad sense. In addition, the apparatus 100 may be implemented by one or more virtual computer environments executable in a computer. Alternatively, the apparatus 100 may be a dedicated computer designed for training an estimation model which estimates a feature value of an object or estimating the feature value of the object using the estimation model, or may be dedicated hardware realized by a dedicated circuit.

Regarding the function of training the estimation model, the apparatus 100 acquires, for each of a plurality of groups, training data including respective images of a plurality of objects and group labels assigned to the groups. By using training data in which an individual object is not assigned a label, the apparatus 100 trains the estimation model which estimates, when an image of an individual object is input, a feature value of the object. The apparatus 100 adopts at least one of following two types of approaches in order to train an estimation model which estimates a feature value of an individual object by using a group label indicating a feature value of an entire group.

### 1 Approach 1

The apparatus 100 generates a training feature value for an individual object in a group, based on an image of each object. The apparatus 100 performs training processing of the estimation model by using the feature value of the individual object generated for training.

### 2 Approach 2

The apparatus 100 combines or integrates feature values of individual objects which are output by the estimation model in response to inputs of images of objects included in the training data, and calculates an estimation value of a group label. The apparatus 100 performs training processing of the estimation model such that the estimation value of the group label is brought close to the group label.

Hereinafter, the apparatus 100 will be described in more detail.

Regarding the function of estimating the feature value of the object using the estimation model, the apparatus 100 includes an image acquisition unit 110, an estimation unit 120, and an output unit 130. When the apparatus 100 has a function of performing training processing of an estimation model which estimates a feature value of an object, the apparatus 100 may include a label acquisition unit 140, a training data storage unit 145, a first calculation unit 150, a second calculation unit 180, and a training processing unit 190. In the present embodiment, description will be given on assumption that the apparatus 100 has both the function of performing the training processing of the estimation model which estimates the feature value of the object and the function of estimating the feature value of the object using the estimation model.

The image acquisition unit 110 is communicably connected to the image capturing apparatus 20, and acquires an image of an object captured by the image capturing apparatus 20 using wired communication or wireless communication. The image acquisition unit 110 according to the present embodiment acquires an image of each object included in a sample from the image capturing apparatus 20 which is a flow imaging apparatus.

The image acquisition unit 110 may acquire images of all objects included in a group (for example, a sample) of a plurality of objects, or may acquire images of some objects included in the sample. Even when the apparatus 100 acquires images of some objects included in the sample, increasing a number of at least one object sampled can further increase an accuracy of the estimation model to be trained.

The estimation unit 120 is connected to the image acquisition unit 110. In response to an input of an image of an evaluation target object, the estimation unit 120 outputs an estimation value of a feature value (also referred to as an “individual feature value”) of the evaluation target object. The estimation unit 120 includes an estimation model 125. The estimation model 125 is a model which, when an image of an individual object to be evaluated is input, outputs an estimation value of an individual feature value of the object. The estimation unit 120 uses the estimation model 125, and inputs an image of an evaluation target object to the estimation model 125 and causes the estimation model 125 to output an estimation value of an individual feature value for the evaluation target object.

The estimation model 125 may be any machine learning model to which an image is input and which outputs one or more values, and may be, for example, a neural network such as a convolutional neural network (CNN), a support vector machine (SVM), or another machine learning model. In such a machine learning model, an internal parameter can be adjusted such that output data to be output according to input data for training is brought close to a target value, whereby training can be performed so as to output desired output data according to the input data.

The output unit 130 is connected to the estimation unit 120. The output unit 130 outputs the estimation value of the individual feature value estimated by the estimation unit 120, as an evaluation value of the individual feature value for the evaluation target object. Accordingly, by using an image of an individual object included in a group of a plurality of objects, the output unit 130 can output an evaluation value obtained by evaluating the individual object.

The label acquisition unit 140 acquires, as a label for training, a group label assigned to a group including a plurality of objects. The label acquisition unit 140 may be an input apparatus, and may acquire the group label upon receiving an input of an observation result or an analysis result by an observer, an analyst, or the like of a sample. When the sample is analyzed by an analysis apparatus or the like, the label acquisition unit 140 may acquire, as a group label, an analysis result by the analysis apparatus.

The group label is an evaluation value or a feature value allocated to the entire group. The group label may be a numerical value such as a real number or an integer, or may be a flag, a tag, or the like indicating presence or absence of a predetermined property or the like. When the object is a cell or a microorganism, the group label may be a measurement result, an evaluation result, or the like of any property or feature value possessed by an entire sample, and the individual feature value may be an estimation value or an evaluation value of the property or feature value possessed by an individual cell or microorganism. In the present embodiment, the group label is, as an example, a photosynthesis activity value of the entire sample, and the individual feature value may be an estimation value or an evaluation value of photosynthesis activity of the individual object (cell or microorganism).

The training data storage unit 145 is connected to the image acquisition unit 110 and the label acquisition unit 140. The training data storage unit 145 receives, from the image acquisition unit 110 and the label acquisition unit 140, each image of a plurality of objects included in a group and a group label assigned to the group, and stores them. The training data storage unit 145 may store, as training data, a data set including an image of each object and a group label for each of one or more groups.

The first calculation unit 150 is connected to the training data storage unit 145. The first calculation unit 150 uses an image and a group label of each of a plurality of objects for a group applied to training of the estimation model 125 to calculate an individual feature value of each of the plurality of objects. Accordingly, for training data in which only one group label is assigned to an entire group, the first calculation unit 150 can assign an individual feature value of each object in the group, and can prepare training data including a set of an image and an individual feature value for each object.

The first calculation unit 150 includes a morphological index value calculation unit 155, a morphological feature value calculation unit 160, and an individual feature value calculation unit 170. For each of a plurality of objects in a group, the morphological index value calculation unit 155 calculates, from an image of each object, one or more morphological index values indicating at least one of morphology, color, or markings of the object. As an example, the morphological index value may be a predetermined parameter value that can be calculated from appearance of an object, such as, for example, a size or circularity of the object, or another morphological or shape characteristic of the object; luminance, hue, saturation, lightness, or another color characteristic of the object; or a pattern, texture, visual texture, or another marking characteristic of the object. The morphological index value may be a numerical value, a sign representing a shape or the like, or the like. The morphological index value calculation unit 155 may include a circuit or a program for calculating such a morphological index value, and calculates a morphological index value by inputting an image of an object and outputs the calculated morphological index value.

For each of a plurality of objects in the group, the first calculation unit 150 calculates, by the morphological feature value calculation unit 160 and the individual feature value calculation unit 170, an individual feature value using one or more morphological index values of the object and a group label. For each of the plurality of objects, the morphological feature value calculation unit 160 calculates one or more morphological feature values using one or more morphological index values. The morphological feature value calculation unit 160 may include a first calculation model 165. The first calculation model 165 receives an input of one or two or more morphological index values, and calculates morphological feature values corresponding to these morphological index values.

The individual feature value calculation unit 170 is connected to the morphological feature value calculation unit 160. For each of the plurality of objects, the individual feature value calculation unit 170 calculates an individual feature value by using one or more morphological feature values. The individual feature value calculation unit 170 may include a second calculation model 175 and calculate an individual feature value of the object by the second calculation model 175. The second calculation model 175 receives an input of the one or more morphological feature values for the object calculated by the morphological feature value calculation unit 160, and combines the one or more morphological feature values to calculate the individual feature value of the object.

The second calculation unit 180 is connected to the estimation unit 120. For a plurality of objects, the second calculation unit 180 calculates an estimation value of a group label of the group including the plurality of objects by using estimation values of a plurality of individual feature values estimated for respective objects by the estimation model 125. The second calculation unit 180 may calculate the estimation value of the group label from the estimation values of the plurality of individual feature values, in accordance with a known relationship between individual feature values of a plurality of objects in a group and a feature value of the entire group. Note that, when the estimation value of the group label is not used for training, the apparatus 100 may not include the second calculation unit 180.

The training processing unit 190 is connected to the estimation unit 120, the first calculation unit 150, and the second calculation unit 180. For each group, the training processing unit 190 performs training processing of the estimation model 125 by using respective images and individual feature values of a plurality of objects. In the training processing, the training processing unit 190 may update a learnable parameter in the estimation model 125 such that the estimation value of the individual feature value which is output by the estimation unit 120 in response to an input of an image of an object to be learned to the estimation unit 120 is brought close to the individual feature value calculated for this object by the first calculation unit 150 (approach 1). For each group, the training processing unit 190 may perform the training processing of the estimation model 125 such that the estimation value of the group label calculated by the second calculation unit 180 is brought close to the group label stored in the training data storage unit 145 (approach 2).

In the present disclosure, the morphological index value calculation unit 155, the morphological feature value calculation unit 160, and the individual feature value calculation unit 170 described above are “calculation units” which calculate values, parameters, or data, and, for convenience of explanation, are expressed in the present disclosure as names to which names of values, parameters, or data to be calculated ("morphological index value", "morphological feature value", and "individual feature value") are appended. Similarly to the first calculation unit 150 and the second calculation unit 180, the morphological index value calculation unit 155, the morphological feature value calculation unit 160, and the individual feature value calculation unit 170 may be referred to as a "third calculation unit", a "fourth calculation unit", a "fifth calculation unit", or the like. Here, words "first", "second", "third", "fourth", and "fifth" are given formally to distinguish the calculation units from one another, and do not mean that there is any fixed order. Therefore, when referring to each calculation unit of the first calculation unit 150, the second calculation unit 180, the morphological index value calculation unit 155, the morphological feature value calculation unit 160, and the individual feature value calculation unit 170, “first”,“ second”, “third”, “fourth”, “fifth”, or the like attached to each calculation unit may be appropriately interchanged depending on an order of reference or other circumstances.

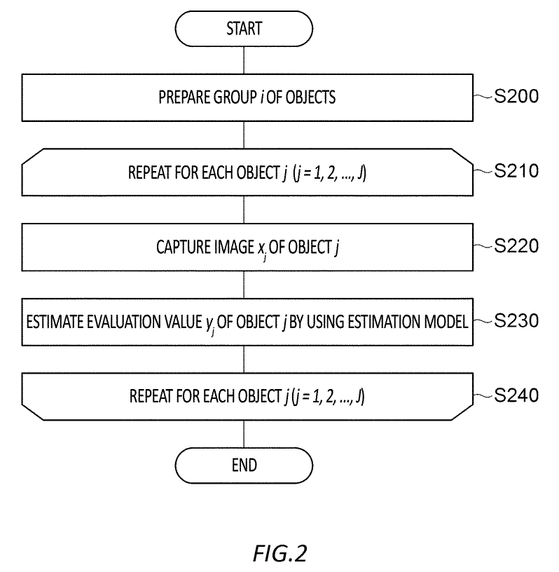

FIG. 2 illustrates an evaluation processing flow of the system 10 according to the present embodiment. According to the evaluation processing flow of the present drawing, the system 10 receives an input of an image of each object in a group and outputs an evaluation value of each object.

In S200 (step 200), a group i including a plurality of objects is prepared. Here, i is an integer of 1 or 2 or more (i = 1, 2, ..., I). In the present embodiment, a sample as a cell group including a plurality of cells is prepared as the group i including a plurality of objects.

In S210 to S240, the apparatus 100 repeats processing from S220 to S230 for each object in the group i. In the example of the present drawing, the apparatus 100 performs the processing of S220 to S230 on each object j (j = 1, 2, ..., J) of J objects in the group i. Here, J is a number of at least one object included in the group i, and is an integer of 1 or 2 or more. Note that the number J of at least one object included in the group i may differ depending on the group i. In addition, the evaluation apparatus 100 may perform the processing from S220 to S230 only for some of the objects j in the group i.

In S220, the image capturing apparatus 20 captures an image xⱼ of the object j to be evaluated. The image acquisition unit 110 acquires the image xⱼ of the object j from the image capturing apparatus 20 and supplies the image xⱼ to the estimation unit 120.

In S230, the estimation unit 120 outputs an estimation value of an individual feature value for the evaluation target object j by using the estimation model 125 in response to an input of the image xⱼ of the evaluation target object j. The output unit 130 outputs, as an evaluation value y j of the evaluation target object j, the estimation value of the individual feature value estimated by the estimation unit 120. The apparatus 100 repeats the processing from S220 to S230 for each object j.

According to the apparatus 100 described above, the individual feature value of the object can be estimated from the image of the individual object by using the estimation model 125 trained using the image of each object included in the group and a group label assigned to the entire group, and output as the evaluation value. Note that, instead of acquiring the image of each object j from the image capturing apparatus 20, the apparatus 100 may receive supply of a captured image regarding the object j targeted for evaluation value estimation and output the evaluation value of the individual feature value of the object j.

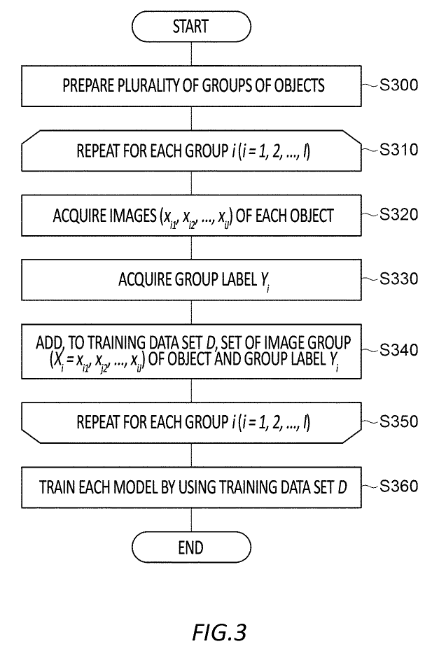

FIG. 3 illustrates an overall flow of training processing of the system 10 according to the present embodiment. In step 300 (S300), a plurality of groups i are prepared. A combination of objects included in the group i and a ratio of each object may be different for each group i.

In S310 to S350, processing from S320 to S340 is repeated for each group among the plurality of groups. In an example of the present drawing, the processing of S320 to S340 are performed for each group i (i = 1, 2, ..., I) of I groups. Here, I is a number of at least one group and is an integer of 1 or 2 or more.

In S320, the image capturing apparatus 20 captures images xᵢ₁, x ᵢ₂, ..., x_{iJ} of each object j included in the group i. The image acquisition unit 110 acquires the images xᵢ₁, xᵢ₂, ..., x_{iJ} of each object from the image capturing apparatus 20.

In S330, the label acquisition unit 140 acquires a group label Y i for the group i. Here, as the group label Y i for the group i, a feature value that can be observed from the group i may be allocated as a result of measurement, experiment, analysis, or the like regarding the group i.

In S340, the image acquisition unit 110 and the label acquisition unit 140 store, in the training data storage unit 145, a set of the images xᵢ₁, xᵢ₂, ..., x_{iJ} of each object included in the group i and the label Y i of the group i. Accordingly, the image acquisition unit 110 and the label acquisition unit 140 add training data for the group i to a training data set D in the training data storage unit 145. The apparatus 100 repeats the processing from S320 to S340 for each group i.

In S360, the training processing unit 190 performs training processing of training the estimation model 125 by using the training data set D stored in the training data storage unit 145. The training processing unit 190 may further perform training processing of the second calculation model 175 by using the training data set D. The training processing unit 190 may update the model to be trained, by performing further training on the already trained estimation model 125 or the second calculation model 175 by using a new training data set D.

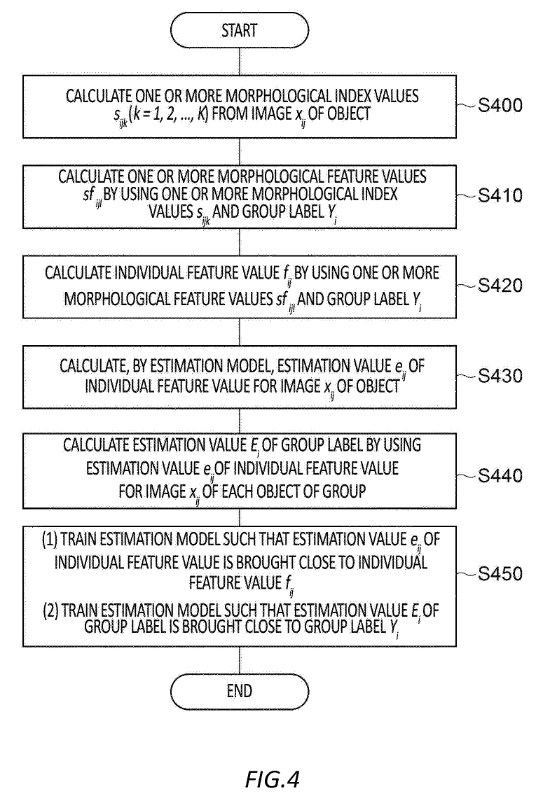

FIG. 4 is a detailed flow of the training processing of the system 10 according to the present embodiment. The system 10 may perform, based on the flow of the present drawing, the training processing using training data regarding each group i among a plurality of groups in S360 of FIG. 3.

In S400, the morphological index value calculation unit 155 calculates, for each of a plurality of objects j in the group i, one or more morphological index values s ijk (k = 1, 2, ..., K) indicating at least one of morphology, color, or markings of the object j from an image x ij of each object j. Here, K is a number of at least one morphological index value calculated from the image of the object, and is an integer of 1 or 2 or more.

In S410, the morphological feature value calculation unit 160 calculates one or more morphological feature values sf ijl (l = 1, 2, ..., L) by using the one or more morphological index values s ijk for each of the plurality of objects j in the group i. The morphological feature value calculation unit 160 may calculate one or more morphological feature values sf ijl by further using the group label Y i. The morphological feature value calculation unit 160 may calculate one or more morphological feature values sf ijl by the first calculation model 165.

The first calculation model 165 receives an input of one or two or more morphological index values s ijk and calculates the morphological feature value sf ijl corresponding to these morphological index values s ijk. For each of one or more morphological feature values sf ijl to be calculated, the first calculation model 165 may receive an input of one or two or more morphological index values s ijk used for calculating the morphological feature value sf ijl and calculate the morphological feature value sf ijl. Here, the morphological feature value sf ijl is a feature value, estimated from the morphology of the object, calculated using one or two or more morphological index values s ijk by a predetermined method. Two morphological feature values different from each other may be calculated from sets of morphological index values different from each other, or may be calculated from sets of morphological index values having at least some morphological index values in common.

The first calculation model 165 may be realized by utilizing, for example, a relational expression or a prediction expression between the morphological index value and the individual feature value of the object that has been found to affect the individual feature value of the object, which has been clarified by knowledge of experts, analysis results, or the like, or a relational expression or a prediction expression between the morphological index value and the individual feature value obtained from a variation or distribution of the morphological index value and the individual feature value of the object. When the first calculation model 165 implements such a relational expression or prediction expression and receives an input of one or two or more morphological index values s ijk, the first calculation model 165 calculates the morphological feature value sf ijl by using such a relational expression or prediction expression.

Such a relational expression or prediction expression may be created by using a relationship between an evaluation value or a feature value measured for each of a plurality of samples that may include one or two or more types of objects and a statistic (average value, median value, maximum value, minimum value, or the like) of morphological index values of a plurality of objects in each sample. The morphological feature value calculation unit 160 may generate the first calculation model 165 by using the training data stored in the training data storage unit 145.

Specifically, the morphological feature value calculation unit 160 receives morphological index values s ijk of a plurality of objects j in each group i stored in the training data storage unit 145 via the morphological index value calculation unit 155, and calculates a statistic of the morphological index values s ijk of the objects j in the group i. In addition, the morphological feature value calculation unit 160 receives the group label Y i of each group i stored in the training data storage unit 145. The morphological feature value calculation unit 160 generates a relational expression between a statistic of one or two or more morphological index values s ijk for the group i and the group label Y i, and registers the relational expression as the first calculation model 165. The morphological feature value calculation unit 160 may use linear regression, Bayesian linear regression, simple regression, multiple regression, Lasso regression, elastic net regression, ridge regression, support vector regression, Gaussian process regression, or other regression to generate a regression expression having the statistic of the morphological index values s ijk as an explanatory variable and the group label Y i, which is the feature value of the entire group i, as an objective variable, and use the regression expression as the relational expression between the statistic of the morphological index values s ijk and the group label Y i for the group i. Here, the first calculation model 165 may select only a regression expression for a combination of the statistic of one or two or more morphological index values s ijk and the group label Y i in which a correlation between them exceeds a threshold that is designated or set in advance and use the selected regression expression as the first calculation model 165.

The morphological feature value calculation unit 160 may calculate one or more morphological feature values sf ijl by using one or more morphological index values s ijk and the group label Y i for each of the plurality of objects j in the group i. For example, when the individual feature value of the object j is affected by the feature value of the entire group, the first calculation model 165 may use the group label Y i as one of variables (explanatory variables) of a relational expression for calculating the morphological feature value sf ijl from the morphological index value s ijk.

In S420, for each of the plurality of objects j in the group i, the individual feature value calculation unit 170 calculates an individual feature value f ij by using one or more morphological feature values sf ijl. The individual feature value calculation unit 170 may calculate the individual feature value f ij of the object j by the second calculation model 175. Note that when the second calculation model 175 is not updated, the individual feature value calculation unit 170 may store, in the training data storage unit 145, the individual feature value f ij calculated for each object j in the group i. Accordingly, the apparatus 100 can repeatedly use the individual feature values f ij stored in the training data storage unit 145 without recomputing the individual feature values f ij.

The apparatus 100 may not include the individual feature value calculation unit 170. In this case, the apparatus 100 may supply, as an individual feature value, one morphological feature value output by the morphological feature value calculation unit 160 to the training processing unit 190 without performing the processing of S420.

Here, the morphological feature value calculation unit 160 can calculate a plurality of types of morphological feature values sf ijl according to various knowledge of experts, various analysis results, generation of one or two or more relational expressions by, for example, the morphological feature value calculation unit 160, and/or the like. The second calculation model 175 combines a plurality of types of morphological feature values sf ijl calculated for the object and outputs a result as an individual feature value of the object. The second calculation model 175 may calculate the individual feature value by weighted combination (a weighted sum or the like) of the plurality of morphological feature values sf ijl. In addition, the second calculation model 175 may calculate the individual feature value by further using a group label. In this case, the second calculation model 175 calculates the individual feature value by weighted combination of one or more morphological feature values sf ijl and the group label Y i. For example, the second calculation model 175 may use a morphological feature value sf ij1, a morphological feature value sf ij2, ..., a morphological feature value sf ijL, and the group label Y i to calculate the individual feature value f ij by a weighted sum represented by the individual feature value f ij= α 1×sf ij1+ α 2 ×sf ij2+ ... + α L×sf ijL+ βY i (here, α 1, α 2, ..., α L, and β are weights of the respective morphological feature values sf ijl and the group label Y i). Accordingly, the individual feature value calculation unit 170 can integrate one or more morphological feature values sf ijl and the group label Y i, and output the individual feature value f ij usable for training of the estimation model 125.

In S430, for each of the plurality of objects j in the group i, the estimation unit 120 calculates, by the estimation model 125, an estimation value e ij obtained by estimating the individual feature value f ij of the object j from the image x ij of the object j.

In S440, the second calculation unit 180 calculates an estimation value E i of the group label Y i of the group i by using the estimation value e ij of the individual feature value f ij estimated by the estimation model 125 for each of the plurality of objects j in the group i. The second calculation unit 180 may calculate, as the estimation value E i of the group label Y i, a statistic (average value, median value, maximum value, minimum value, or the like) of the estimation values e ij of the plurality of individual feature values f ij, for example, in accordance with a known relationship between the individual feature value f ij of each object j of the group i and the feature value of the entire group i. For example, when a value indicating an activity degree of cells or microorganisms, such as a photosynthesis activity value, for the entire group i of samples or the like is used as the group label Y i, the second calculation unit 180 may set, as the estimation value E i of the group label Y i, an average of the estimation values e ij of the individual feature values (activity values) of the objects (cells) in the group i.

Alternatively, the second calculation unit 180 may calculate a feature value for the entire group when a plurality of objects j each having the estimation value e ij of the individual feature value estimated by the estimation unit 120 are present in the group i, and may set the feature value as the estimation value E i of the group label. For example, when a biodegradation amount for the entire group i is used as the group label Y i, the second calculation unit 180 may set, as the estimation value E i of the group label Y i, a sum of the estimation values e ij of the individual feature values (biodegradation amounts) of the objects (cells) in the group i.

In S450, the training processing unit 190 performs training processing of the estimation model 125 related to the group i. The training processing unit 190 may perform the training processing of the estimation model 125 by any one or both of following methods.

### 1 Training using estimation value e ij of individual feature value of each object j (approach 1)

For the group i, the training processing unit 190 performs the training processing of the estimation model 125 such that the estimation value e ij of the individual feature value output by the estimation unit 120 in response to an input of the image x ij of the object j to be learned to the estimation unit 120 is brought close to the individual feature value f ij calculated by the first calculation unit 150 for the object j. The training processing unit 190 updates the learnable parameter in the estimation model 125 by the training processing. For example, when a neural network is used as the estimation model 125, the training processing unit 190 adjusts a weight between neurons of the neural network, a bias of each neuron, or the like, using an error of the estimation value e ij of the individual feature value output by the neural network in response to an input of the image x ij of the object j with respect to the individual feature value f ij calculated by the first calculation unit 150, by a method such as back propagation. The training processing unit 190 may train the estimation model 125 by using a known learning algorithm for a machine learning model adopted as the estimation model 125. The training processing unit 190 may repeat the training processing illustrated in the present drawing until an error such as a root mean squared percentage error (RMSPE) between the estimation value e ij of the individual feature value calculated using at least a part of the training data and the individual feature value f ij becomes less than or equal to a predetermined threshold value or training for a predetermined training time or a predetermined number of times of training has been completed.

According to this method, even when the feature value of the entire group is known but the individual feature value of each object is unknown, the system 10 can generate the individual feature value of each object by using the first calculation model 165 or the second calculation model 175 in addition to the first calculation model 165 and use the generated individual feature value for training the estimation model 125. Therefore, a user of the system 10 can reduce a workload of labeling each object included in the group.

Since the system 10 can perform training processing reflecting prior knowledge such as various knowledge or analysis results, prior distribution, or the like, it is possible to efficiently construct the estimation model 125 and improve explainability of the system 10. Since the system 10 does not need to store training data individually labeled for each object, a size of the training data can be reduced.

### 2 Training using group label Y i assigned to group i (approach 2)

The training processing unit 190 performs the training processing of the estimation model 125 such that the estimation value E i of the group label calculated by the second calculation unit 180 from the estimation value e ij of the individual feature value of each object j is brought close to the group label Y i. The training processing unit 190 updates the learnable parameter in the estimation model 125 by the training processing. For example, when a neural network is used as the estimation model 125, the training processing unit 190 may calculate an error of the estimation value e ij of the individual feature value with respect to the individual feature value f ij by distributing, to a plurality of objects, an error of the estimation value E i of the group label calculated by the second calculation unit 180 with respect to the group label Y i, and adjust a parameter of the neural network in a manner similar to in (1) above. When the training processing unit 190 distributes an estimation error for the entire group to an estimation error for each object, the error of the estimation value of the feature value of the entire group may be used as the error of the individual feature value of each object, for example, by equally dividing the error or by using it as it is, in accordance with a relationship between the feature value of the entire group and the individual feature value of each object. The training processing unit 190 may repeat the training processing illustrated in the present drawing until an error such as a root mean squared percentage error (RMSPE) between the estimation value E i of the group label calculated using at least a part of the training data and the group label Y i becomes less than or equal to a predetermined threshold value or training for a predetermined training time or a predetermined number of training iterations has been completed.

According to this method, even when the feature value of the entire group is known but the individual feature value of each object is unknown, the system 10 can generate the estimation value of the feature value of the entire group from the estimation value of the individual feature value of each object by using the second calculation unit 180 and use the generated estimation value for training the estimation model 125. Since a combination of types of objects included in a group is different for each group, the system 10 can improve the estimation accuracy of the feature value of individual object by performing training using the second calculation unit 180 such that the estimation value of the feature value of the entire group for each group is brought close to the group label. Therefore, the user of the system 10 can reduce the workload of labeling each object included in the group. In addition, since the system 10 does not need to store training data individually labeled for each object, the size of the training data can be reduced.

The training processing unit 190 may further perform the training processing of the second calculation model 175. The training processing unit 190 adjusts a learnable parameter in the second calculation model 175 such that the estimation value e ij of the individual feature value for the object j to be learned is brought close to the individual feature value f ij for the object j. For example, when the second calculation model 175 calculates the individual feature value f ij by a weighted combination of one or more morphological feature values sf ijl (and the group label Y i), a weight of the morphological feature value sf ijl (or the group label Y i) closer to the estimation value e ij of the individual feature value may be further increased, and a weight of the morphological feature value sf ijl (or the group label Y i) farther away from the estimation value e ij of the individual feature value may be further decreased.

For example, when the individual feature value f ij = α 1×sf ij1+ α 2×sf ij2+ ... + α P×sf ijL + βY i, the training processing unit 190 may further increase a weight α l (or β) corresponding to the morphological feature value sf ijl (or the group label Y i) closer to the estimation value e ij of the individual feature value, and may further decrease the weight α l (or β) corresponding to the morphological feature value sf ijl (or the group label Y i) farther away from the estimation value e ij of the individual feature value.

Accordingly, the training processing unit 190 can increase weights of more accurate feature values even when a plurality of types of morphology-based feature value prediction methods having different prediction accuracies are introduced in response to various findings, analysis results, or the like. As a result, the system 10 can improve an accuracy with which the first calculation unit 150 generates the individual feature value.

The training processing unit 190 may perform training processing of the first calculation model 165. The training processing unit 190 adjusts a learnable parameter in the first calculation model 165 such that the estimation value e ij of the individual feature value for the object j to be learned is brought close to the individual feature value f ij for the object j. For example, when the first calculation model 165 is realized by a relational expression or a prediction expression for calculating the morphological feature value sf ijl from the morphological index value s ijk or calculating the morphological feature value sf ijl from the morphological index value s ijk and the group label Y i, the training processing unit 190 may adjust a parameter (a coefficient or the like) of the relational expression or the prediction expression such that the estimation value e ij of the individual feature value or the morphological feature value sf ijl is brought close to the individual feature value f ij.

According to designation or setting of the user or the like, the apparatus 100 may change an update amount (a training rate or the like) of at least one of the estimation model 125, the second calculation model 175, or the first calculation model 165, based on the estimation error of the individual feature value (the error of the estimation value e ij of the individual feature value with respect to the individual feature value f ij). Accordingly, the apparatus 100 can adjust an update speed of at least one of the estimation model 125, the second calculation model 175, or the first calculation model 165, and can adjust how much an update speed of a model based on prior knowledge such as the first calculation model 165 and the second calculation model 175 is increased as compared with the update speed of the estimation model 125.

Various embodiments of the present invention may be described with reference to flowcharts and block diagrams, where blocks may represent (1) stages of processes in which operations are executed or (2) sections of apparatuses responsible for executing operations. Certain stages and sections may be implemented by a dedicated circuit, a programmable circuit supplied together with computer-readable instructions stored on computer readable media, and/or processors supplied together with computer-readable instructions stored on computer readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer readable medium may include any tangible device that can store instructions to be executed by a suitable device, and as a result, the computer readable medium having instructions stored thereon includes a product including instructions that can be executed in order to create means for executing operations specified in the flowcharts or block diagrams. Examples of the computer readable medium may include an electronic storage medium, a magnetic storage medium, an optical storage medium, an electromagnetic storage medium, a semiconductor storage medium, and the like. More specific examples of the computer readable medium may include a FLOPPY (registered trademark) disk, a diskette, a hard disk, a random access memory (RAM), a read only memory (ROM), an erasable programmable read only memory (EPROM or a flash memory), an electrically erasable programmable read only memory (EEPROM), a static random access memory (SRAM), a compact disc read only memory (CD-ROM), a digital versatile disk (DVD), a BLU-RAY (registered trademark) disk, a memory stick, an integrated circuit card, and the like.

A computer-readable instruction may include: an assembler instruction, an instruction-set-architecture (ISA) instruction; a machine instruction; a machine dependent instruction; a microcode; a firmware instruction; state-setting data; or either a source code or an object code described in any combination of one or more programming languages, including an object oriented programming language such as SMALLTALK (registered trademark), JAVA (registered trademark), C++, or the like, and a conventional procedural programming language such as a "C" programming language or a similar programming language.

The computer-readable instruction may be provided for a processor or programmable circuit of a programmable data processing apparatus, such as a computer, locally or via a local area network (LAN), a wide area network (WAN) such as the Internet, or the like to execute the computer-readable instruction in order to create means for executing the operations specified in the flowcharts or block diagrams. Here, the computer may be a personal computer (PC), a tablet computer, a smartphone, a workstation, a server computer, or a computer such as a general purpose computer or a special purpose computer, or may be a computer system to which a plurality of computers are connected. Such computer system to which the plurality of computers are connected is also referred to as a distributed computing system, and is a computer in a broad sense. In a distributed computing system, a plurality of computers collectively execute a program by each of the plurality of computers executing a part of the program, and passing data during the execution of the program among the computers as needed.

Examples of the processor include a computer processor, a central processing unit (CPU), a processing unit, a microprocessor, a digital signal processor, a controller, a microcontroller, and the like. The computer may include one processor or a plurality of processors. In a multiprocessor system including a plurality of processors, the plurality of processors collectively execute a program by each of the processors executing a part of the program, and passing data during the execution of the program among the processors as needed. For example, in execution of multiple tasks, each of the plurality of processors may execute a portion of each task pieces by pieces by performing task-switching for each time slice. In this case, which portion of one program each processor is responsible for executing dynamically changes. Moreover, which portion of the program each of the plurality of processors is responsible for executing may be determined statically by multiprocessor-aware programming.

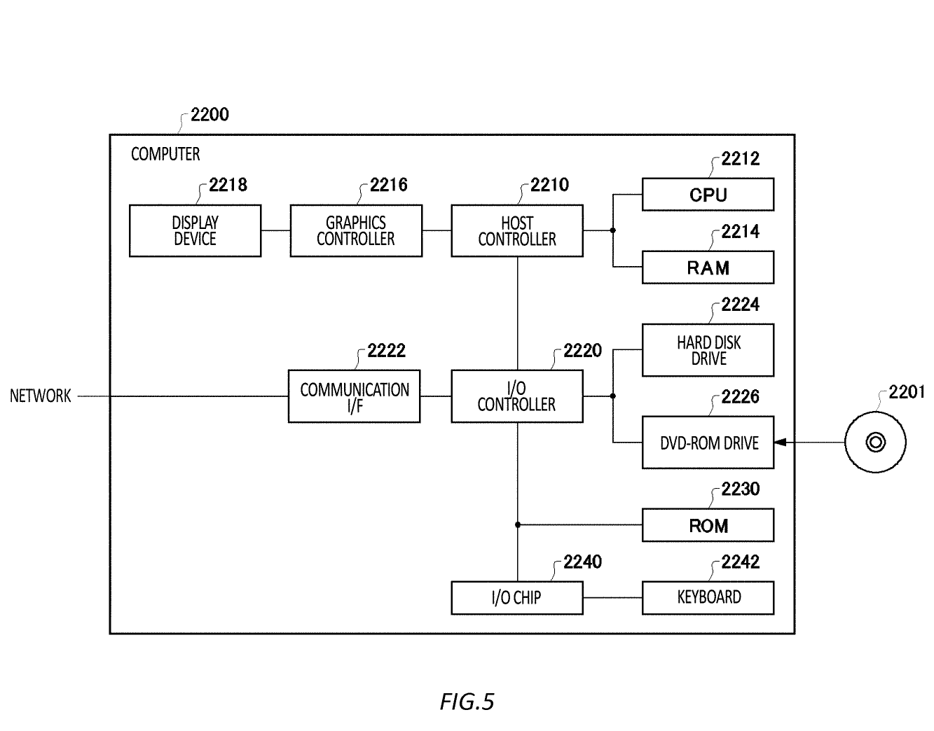

FIG. 5 illustrates an example of a computer 2200 in which a plurality of aspects of the present invention may be embodied in whole or in part. A program installed in the computer 2200 can cause the computer 2200 to function as an operation associated with the apparatuses according to the embodiments of the present invention or as one or more sections of the apparatuses, or can cause the operation or the one or more sections to be executed, and/or can cause the computer 2200 to execute a process according to the embodiments of the present invention or a stage of the process. Such a program may be performed by a CPU 2212 so as to cause the computer 2200 to perform certain operations associated with some or all of the blocks of flowcharts and block diagrams described in the present specification.

The computer 2200 according to the present embodiment includes the CPU 2212, an RAM 2214, a graphics controller 2216, and a display device 2218, which are mutually connected by a host controller 2210. The computer 2200 also includes a communication interface 2222, a storage apparatus such as a hard disk drive 2224, and input/output units such as a DVD-ROM drive 2226 and an IC card drive, which are connected to the host controller 2210 via an input/output controller 2220. The computer also includes legacy input/output units such as an ROM 2230 and a keyboard 2242, which are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 in a frame buffer or the like provided in the RAM 2214 or in itself, such that the image data is displayed on the display device 2218.

The communication interface 2222 communicates with other electronic devices via a network. The storage apparatus such as the hard disk drive 2224 stores programs and data to be used by the CPU 2212 in the computer 2200. The DVD-ROM drive 2226 reads programs or data from a DVD-ROM 2201 and provides the programs or data to the storage apparatus such as the hard disk drive 2224 via the RAM 2214. The IC card drive reads programs and the data from the IC card, and/or writes the programs and the data to the IC card.

The ROM 2230 stores therein boot programs and the like executed by the computer 2200 at the time of activation, and/or programs that depend on the hardware of the computer 2200. The input/output chip 2240 may also connect various input/output units to the input/output controller 2220 via a parallel port, a serial port, a keyboard port, a mouse port, or the like.

Programs are provided by a computer readable medium such as the DVD-ROM 2201 or the IC card. The programs are read from the computer readable medium, installed on the storage apparatus such as the hard disk drive 2224, the RAM 2214 or the ROM 2230, which are also examples of the computer readable medium, and executed by the CPU 2212. The information processing described in these programs is read by the computer 2200 and provides cooperation between the programs and the above-described various types of hardware resources. The apparatus or method may be configured by implementing operations or processing of information according to use of the computer 2200.

For example, when communication is performed between the computer 2200 and an external device, the CPU 2212 may execute a communication program loaded in the RAM 2214 and instruct the communication interface 2222 to perform communication processing based on a processing written in the communication program. The communication interface 2222 reads transmission data stored in a transmission buffer processing region provided on the RAM 2214, the storage apparatus such as the hard disk drive 2224, the DVD-ROM 2201, or a recording medium such as an IC card under the control of the CPU 2212, transmits the read transmission data to the network, or writes reception data received from the network to a reception buffer processing region or the like provided on a recording medium.

In addition, the CPU 2212 may cause the RAM 2214 to read all or a necessary part of a file or database stored in the storage apparatus such as the hard disk drive 2224, the DVD-ROM drive 2226 (DVD-ROM 2201), or an external recording medium such as an IC card, and may execute various types of processing on data on the RAM 2214. Then, the CPU 2212 writes the processed data back in the external recording medium.

Various types of information such as various types of programs, data, tables, and databases may be stored in a recording medium and subjected to information processing. The CPU 2212 may execute, on the data read from the RAM 2214, various types of processing including various types of operations, information processing, conditional judgement, conditional branching, unconditional branching, information retrieval/replacement, or the like described throughout the present disclosure and specified by instruction sequences of the programs, and writes the results back to the RAM 2214. In addition, the CPU 2212 may retrieve information in a file, a database, or the like in the recording medium. For example, when a plurality of entries, each having an attribute value of a first attribute associated with an attribute value of a second attribute, are stored in the recording medium, the CPU 2212 may retrieve, out of the plurality of entries, an entry with the attribute value of the first attribute specified that meets a condition, read the attribute value of the second attribute stored in said entry, thereby acquiring the attribute value of the second attribute associated with the first attribute meeting a predetermined condition.

The programs or software modules described above may be stored in a computer readable medium on or near the computer 2200. In addition, a recording medium such as a hard disk or an RAM provided in a server system connected to a dedicated communication network or the Internet can be used as the computer readable medium, thereby providing a program to the computer 2200 via the network.

While the present invention has been described above by way of the embodiments, the technical scope of the present invention is not limited to the scope described in the above-described embodiments. It is apparent to persons skilled in the art that various alterations or improvements can be made to the above-described embodiments. It is also apparent from the description of the claims that the form to which such alterations or improvements are made can be included in the technical scope of the present invention.

It should be noted that the operations, procedures, steps, stages, and the like of each process performed by an apparatus, system, program, and method shown in the claims, the specification, or the drawings can be realized in any order as long as the order is not indicated by “prior to,” “before,” or the like and as long as the output from a previous process is not used in a later process. Even if the operation flow is described by using phrases such as "first" or "next" for the sake of convenience in the claims, specification, and drawings, it does not necessarily mean that the process must be performed in this order. According to the present disclosure, following items are also disclosed.

(Item 1)

An apparatus including:

a first calculation unit which calculates an individual feature value of each of a plurality of objects by using an image of each of the plurality of objects and a group label assigned to a group including the plurality of objects; and

a training processing unit which performs training processing of an estimation model which outputs, when an image of an object is input, an estimation value of the individual feature value of the object by using the image and the individual feature value of each of the plurality of objects.

(Item 2)

The apparatus according to item 1, wherein

the first calculation unit

calculates, for each of the plurality of objects, a morphological index value indicating at least one of morphology, color, or markings of the object from an image of the object, and

calculates, for each of the plurality of objects, the individual feature value by using the morphological index value and the group label.

(Item 3)

The apparatus according to item 1 or 2, wherein

the first calculation unit

calculates, for each of the plurality of objects, one or more morphological index values, including the morphological index value, indicating at least one of morphology, color, or markings of the object from an image of the object,

calculates, for each of the plurality of objects, one or more morphological feature values using the one or more morphological index values by a first calculation model, and

calculates, for each of the plurality of objects, the individual feature value using the one or more morphological feature values by a second calculation model.

(Item 4)

The apparatus according to item 3, wherein the second calculation model calculates the individual feature value by a weighted combination of the one or more morphological feature values and the group label.

(Item 5)

The apparatus according to item 3 or 4, wherein the training processing unit further performs training processing of the second calculation model.

(Item 6)

The apparatus according to any one of items 1 to 5, including

a second calculation unit which calculates, for the plurality of objects, an estimation value of a group label of a group including the plurality of objects by using estimation values of a plurality of individual feature values, including the individual feature value, estimated for respective objects by the estimation model, wherein

the training processing unit performs training processing of the estimation model such that the estimation value of the group label is brought close to the group label.

(Item 7)

The apparatus according to item 6, wherein the second calculation unit calculates, as the estimation value of the group label, a statistic of the estimation values of the plurality of individual feature values.

(Item 8)

The apparatus according to any one of items 1 to 7, including an estimation unit which outputs, in response to an input of an image of an evaluation target object, the estimation value of the individual feature value for the evaluation target object by using the estimation model.

(Item 9)

The apparatus according to any one of items 1 to 8, wherein each object of the plurality of objects is a cell, and a group including the plurality of objects is a cell group.

(Item 10)

An apparatus including:

an estimation unit which estimates an individual feature value of each of a plurality of objects by an estimation model which outputs, when an image of an object is input, an estimation value of the individual feature value of the object;

a second calculation unit which calculates, for the plurality of objects, an estimation value of a group label assigned to a group including the plurality of objects by using estimation values of a plurality of individual feature values, including the individual feature value, estimated for respective objects by the estimation model; and

a training processing unit which performs training processing of the estimation model such that the estimation value of the group label output by the estimation model is brought close to the group label.

(Item 11)

A method including:

calculating, by a computer, an individual feature value of each of a plurality of objects by using an image of each of the plurality of objects and a group label assigned to a group including the plurality of objects; and

performs, by the computer, training processing of an estimation model which estimates, when an image of an object is input, an estimation value of the individual feature value of the object by using the image and the individual feature value of each of the plurality of objects.

(Item 12)

A method including:

estimating, by a computer, an individual feature value of each of a plurality of objects by an estimation model which outputs, when an image of an object is input, an estimation value of the individual feature value of the object;

calculating, by the computer, for the plurality of objects, an estimation value of a group label assigned to a group including the plurality of objects by using estimation values of a plurality of individual feature values, including the individual feature value, estimated for respective objects by the estimation model; and

performing, by the computer, training processing of the estimation model such that the estimation value of the group label output by the estimation model is brought close to the group label.

(Item 13)

A program which, when executed by a computer, causes the computer to perform operations including:

calculating an individual feature value of each of a plurality of objects by using an image of each of the plurality of objects and a group label assigned to a group including the plurality of objects; and

performing training processing of an estimation model which estimates, when an image of an object is input, an estimation value of the individual feature value of the object by using the image of each of the plurality of objects and the individual feature value.

(Item 14)

A program which, when executed by a computer, causes the computer to perform operations including:

estimating an individual feature value of each of a plurality of objects by an estimation model which outputs, when an image of an object is input, an estimation value of the individual feature value of the object;

calculating, for the plurality of objects, an estimation value of a group label assigned to a group including the plurality of objects by using estimation values of a plurality of individual feature values, including the individual feature value, estimated for respective objects by the estimation model; and

performing training processing of the estimation model such that the estimation value of the group label output by the estimation model is brought close to the group label.

### EXPLANATION OF REFERENCES

10: system; 20: image capturing apparatus; 100: apparatus; 110: image acquisition unit; 120: estimation unit; 125: estimation model; 130: output unit; 140: label acquisition unit; 145: training data storage unit; 150: first calculation unit; 155: morphological index value calculation unit; 160: morphological feature value calculation unit; 165: first calculation model; 170: individual feature value calculation unit; 175: second calculation model; 180: second calculation unit; 190: training processing unit; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; and 2242: keyboard.

What is claimed is:

1. An apparatus comprising
a processor, wherein
the processor
calculates an individual feature value of each of a plurality of objects by using an image of each of the plurality of objects and a group label assigned to a group including the plurality of objects, and
performs training processing of an estimation model which outputs, when an image of an object is input, an estimation value of the individual feature value of the object by using the image and the individual feature value of each of the plurality of objects.

2. The apparatus according to claim 1, wherein
in calculation of the individual feature value, the processor
calculates, for each of the plurality of objects, a morphological index value indicating at least one of morphology, color, or markings of the object from an image of the object, and
calculates, for each of the plurality of objects, the individual feature value by using the morphological index value and the group label.

3. The apparatus according to claim 2, wherein
in calculation of the individual feature value, the processor
calculates, for each of the plurality of objects, one or more morphological index values, including the morphological index value, indicating at least one of morphology, color, or markings of the object from an image of the object,
calculates, for each of the plurality of objects, one or more morphological feature values using the one or more morphological index values by a first calculation model, and
calculates, for each of the plurality of objects, the individual feature value using the one or more morphological feature values by a second calculation model.

4. The apparatus according to claim 3, wherein the second calculation model calculates the individual feature value by a weighted combination of the one or more morphological feature values and the group label.

5. The apparatus according to claim 3, wherein in the training processing of the estimation model, the processor further performs training processing of the second calculation model.

6. The apparatus according to claim 1, wherein
the processor
calculates, for the plurality of objects, an estimation value of a group label of a group including the plurality of objects by using estimation values of a plurality of individual feature values, including the individual feature value, estimated for respective objects by the estimation model, and
performs training processing of the estimation model such that the estimation value of the group label is brought close to the group label.

7. The apparatus according to claim 6, wherein in calculation of the estimation value of the group label, the processor calculates, as the estimation value of the group label, a statistic of the estimation values of the plurality of individual feature values.

8. The apparatus according to claim 1, wherein in response to an input of an image of an evaluation target object, the processor outputs the estimation value of the individual feature value for the evaluation target object by using the estimation model.

9. The apparatus according to claim 1, wherein each object of the plurality of objects is a cell, and a group including the plurality of objects is a cell group.

10. A method comprising:
calculating, by a computer, an individual feature value of each of a plurality of objects by using an image of each of the plurality of objects and a group label assigned to a group including the plurality of objects; and
performing, by the computer, training processing of an estimation model which estimates, when an image of an object is input, an estimation value of the individual feature value of the object by using the image and the individual feature value of each of the plurality of objects.

11. The method according to claim 10, wherein
in calculation of the individual feature value, the computer
calculates, for each of the plurality of objects, a morphological index value indicating at least one of morphology, color, or markings of the object from an image of the object, and
calculates, for each of the plurality of objects, the individual feature value by using the morphological index value and the group label.

12. The method according to claim 11, wherein
in calculation of the individual feature value, the computer
calculates, for each of the plurality of objects, one or more morphological index values, including the morphological index value, indicating at least one of morphology, color, or markings of the object from an image of the object,
calculates, for each of the plurality of objects, one or more morphological feature values using the one or more morphological index values by a first calculation model, and
calculates, for each of the plurality of objects, the individual feature value using the one or more morphological feature values by a second calculation model.

13. The method according to claim 12, wherein the second calculation model calculates the individual feature value by a weighted combination of the one or more morphological feature values and the group label.

14. The method according to claim 10, wherein
the computer
calculates, for the plurality of objects, an estimation value of a group label of a group including the plurality of objects by using estimation values of a plurality of individual feature values, including the individual feature value, estimated for respective objects by the estimation model, and
performs training processing of the estimation model such that the estimation value of the group label is brought close to the group label.

15. The method according to claim 10, wherein each object of the plurality of objects is a cell, and a group including the plurality of objects is a cell group.

16. A non-transitory computer readable medium having recorded thereon a program which, when executed by a computer, causes the computer to perform operations comprising:
calculating an individual feature value of each of a plurality of objects by using an image of each of the plurality of objects and a group label assigned to a group including the plurality of objects; and
performing training processing of an estimation model which estimates, when an image of an object is input, an estimation value of the individual feature value of the object by using the image and the individual feature value of each of the plurality of objects.

17. The non-transitory computer readable medium according to claim 16, wherein
in calculation of the individual feature value, the computer
calculates, for each of the plurality of objects, a morphological index value indicating at least one of morphology, color, or markings of the object from an image of the object, and
calculates, for each of the plurality of objects, the individual feature value by using the morphological index value and the group label.

18. The non-transitory computer readable medium according to claim 17, wherein
in calculation of the individual feature value, the computer
calculates, for each of the plurality of objects, one or more morphological index values, including the morphological index value, indicating at least one of morphology, color, or markings of the object from an image of the object,
calculates, for each of the plurality of objects, one or more morphological feature values using the one or more morphological index values by a first calculation model, and
calculates, for each of the plurality of objects, the individual feature value using the one or more morphological feature values by a second calculation model.

19. The non-transitory computer readable medium according to claim 16, wherein
the computer
calculates, for the plurality of objects, an estimation value of a group label of a group including the plurality of objects by using estimation values of a plurality of individual feature values, including the individual feature value, estimated for respective objects by the estimation model, and
performs training processing of the estimation model such that the estimation value of the group label is brought close to the group label.

20. The non-transitory computer readable medium according to claim 16, wherein each object of the plurality of objects is a cell, and a group including the plurality of objects is a cell group.
